(19) United States
(12) Patent Application Publication
(10) Pub. No.: US 2005/0187204 A1
(43) Pub. Date: Aug. 25, 2005

(54) Medicinal composition for lowering blood lipid level

(75) Inventors: Tatsuhito Kondo, Tokyo (JP); Ikuo Takagi, Matsudo-shi (JP); Masato Nakayama, Kitakatsushika-gun (JP); Yasuhiro Torizumi, Ryugasaki-shi (JP)

Correspondence Address: FRISHAUF, HOLTZ, GOODMAN & CHICK, PC, 220 5TH AVE FL 16, NEW YORK, NY, 10001-7708, US

(73) Assignee: SANKYO COMPANY, LIMITED, Tokyo (JP)

(21) Appl. No.: 11/045,407

(22) Filed: Jan. 27, 2005

Related U.S. Application Data
(63) Continuation-in-part of application No. PCT/JP03/10028, filed on Aug. 6, 2003.

(30) Foreign Application Priority Data
Aug. 8, 2002 (JP) 2002-231618

(51) Int. Cl. A61K 31/56; A61K 31/401; A61K 31/366; A61K 31/225

(52) U.S. Cl. 514/171; 514/423; 514/460; 514/548

(57) ABSTRACT

A pharmaceutical composition for lowering blood lipid levels which contains an HMG-CoA reductase inhibitor and bile acids. For treatment, the components of the composition can be administered together, as a composition, or separately.

This is a Continuation-in-Part Application of International Application No. PCT/JP2003/010028 filed Aug. 6, 2003, which is incorporated herein by reference in its entirety.

### BACKGROUND OF THE INVENTION

The present invention relates to a medicinal (or pharmaceutical) composition comprising an HMG-CoA reductase inhibitor and bile acids, for lowering blood lipid levels.

Statins are compounds that reduce blood cholesterol levels by specifically and competitively inhibiting HMG-CoA reductase activity in vivo.

Furthermore, it has been known that constituents of bile acids ameliorate cholestasis, dissolve cholesterol gallstones, suppress absorption of cholesterol from the intestinal canal, and the like (see for example, Drugs in Japan Ethical Drugs 2002, Edited by Japan Pharmaceutical Information Center, Tokyo Japan, Jiho, Inc., Tokyo Japan).

On the other hand, some reports have disclosed combination therapies involving a statin and constituents of bile acids. Most of these have stated that the combination therapy was used for the dissolution of cholesterol gallstones. In addition, some other reports have stated that the combination described above was used for therapies against biliary hepatopathy, biliary cirrhosis, cholestatic jaundice, secondary hypercholesterolemia, and the like which were caused by impairment of outflow of bile into the duodenum, although the reasons of the impairment were not clear.

Some cases in which combination therapies involving a statin and constituents of bile acids have been used are listed below with each objective of the use:
1) Dissolution therapy against cholesterol gallstones by co-administration of pravastatin and ursodeoxycholic acid (see for example, Journal of New Remedies & Clinics, vol. 43, No. 1, 1994, p. 101-105).
2) Dissolution therapy against cholesterol gallstones by co-administration of simvastatin and ursodeoxycholic acid (see for example, Medical Consultation & New Remedies, vol. 33, No. 10, 1996, p. 1477-1488).
3) Cholesterol saturation index in bile was incrementally reduced following co-administration of lovastatin and ursodeoxycholic acid (see for example, Gastroenterology, vol. 98, No. 6, 1990, p. 1572-1576).
4) Treatment against cerebrotendinous xanthomatosis and thickening of the Achilles tendon by co-administration of pravastatin and chenodeoxycholic acid (see for example, Proceedings of The Japan Society of Clinical Biochemistry and Metabolism, Vol. 29, 1992, p. 184-185).
5) Suggestion of beneficial effects of co-administered simvastatin and ursodeoxycholic acid against cholestasis (see Gut, Vol. 44, No. 3, 1999, p. 552-556).
6) Treatment against hypercholesterolemia by co-administration of pravastatin and ursodeoxycholic acid (see The Journal of Japan Atherosclerosis Society, Vol. 20, 1992, p. 857).

Thus it is consistently reported that co-administration of statins and bile acids is useful for dissolution therapy against cholesterol gallstones, since cholesterol levels in bile are further reduced. Neither arguments nor data against the consistent observations described above have been reported.

On the other hand, with regard to the effects of co-administration of statins and bile acids on blood lipid levels, results reported in the literature are different from the above evaluation. For example, one study reported that co-administration of statins and bile acids is not beneficial in patients with bile duct system disorders such as gallstones and the like (Journal of New Remedies & Clinics, Vol. 43, No. 1, 1994, p. 101-105; Medical Consultation & New Remedies, Vol. 33 No.10, 1996, p. 1477-1488). On the contrary, opposite results have been reported that the combination therapy is beneficial for patients with these same diseases (J. Gastroenterology Vol. 29, 1994, p. 47-55; Lancet Vol. 336, 1990, p. 1196; The Japanese Journal of Gastroenterology, Vol. 90, 1993, p. 539). Thus it would appear that no consistent conclusion has yet been reached.

Furthermore, 2 conflicting results have been reported in cases of non-familial hypercholesterolemic patients without gallstones and healthy subjects, that is, co-administration of statins and bile acids did not show any beneficial effects on reducing blood lipids levels (Gastroenterology, vol. 98, 1990, p. 1572-1576; Proceedings of The Japan Society of Clinical Biochemistry and Metabolism, Vol.29, 1992, p. 184-185; Gut, Vol. 44, No. 3, 1999, p. 552-556), while it was reported that co-administration of statins and bile acids is beneficial in said patients (The Journal of Japan Atherosclerosis Society, Vol. 20, 1992, p. 857). Thus it would also appear that no consistent conclusion has yet been reached in this area.

From the disclosed reports, it seems difficult to speculate on the effects of statins and bile acids on blood lipids.

### BRIEF SUMMARY OF THE INVENTION

In light of this background, the present inventors concluded that it is possible to determine the effects of co-administration of statins and constituents of bile acids in animal studies alone using well-controlled experimental conditions such as animal strains, ages, contents of foods, and breeding environment and have diligently studied the pharmacological actions of combination treatments using statins and constituents of bile acids.

As a result the present inventors found that combined administration of statins and constituents of bile acids lowered blood lipids, and thus completed the present invention.

Since HMG-CoA reductase inhibitors are often given as long-term treatment regimens, it is desirable that lipid-lowering effects are obtained at low doses. In addition, it is ideal to take efficient treatment by a single administration of an agent in patients with hypercholesterolemia and cholestasis or gallstones, or in animals with hypercholesterolemia and cholestasis or gallstones.

The present invention is
(1) a medicinal composition comprising an HMG-CoA reductase inhibitor and bile acids as active ingredients to lower blood lipid levels,
(2) a medicinal composition according to (1), comprising one or more HMG-CoA reductase inhibitors selected from the group consisting of pravastatin, lovastatin, simvastatin, fluvastatin, rivastatin, atorvastatin, pitavastatin, and rosuvastatin as active ingredients,
(3) a medicinal composition according to (1), in which one or more HMG-CoA reductase inhibitors as active ingredients are simvastatin or atorvastatin,
(4) a medicinal composition according to (1), comprising one or more bile acids selected from the group consisting of ursodeoxycholic acid, chenodeoxycholic acid, deoxycholic acid, cholic acid, bile salts, bile extract, bear bile, and ox gallstone as active ingredients,
(5) a medicinal composition according to (1), comprising a bile acid of ursodeoxycholic acid as an active ingredient,
(6) a medicinal composition according to (1), for use in the prevention or treatment of hypercholesterolemia or atherosclerosis.

In addition, the present invention provides
(7) combination therapies of an HMG-CoA reductase inhibitor and bile acids as active ingredients to lower blood lipids levels by administration of an HMG-CoA reductase inhibitor and bile acids simultaneously or separately at certain time intervals, and
(8) combination therapies of an HMG-CoA reductase inhibitor and bile acids as active ingredients to mitigate blood lipid levels.

Furthermore, the present invention provides
(9) a method to lower blood lipid levels by administration of an HMG-CoA reductase inhibitor and bile acids as active ingredients simultaneously or separately at certain time intervals.

The preferable methods of those described in (9) are
(10) a method according to (9), to prevent or treat diseases caused by high blood lipid levels, and
(11) a method according to (9), to prevent or treat hypercholesterolemia or atherosclerosis.

### DETAILED DESCRIPTION OF THE INVENTION

“HMG-CoA reductase inhibitor”, which is one component of the medicinal composition of the present invention refer to agents that competitively and specifically inhibit 3-hydroxy-3-methyl-glutryl-CoA (HMG-CoA) reductase, which is a rate limiting enzyme in the biosynthesis of cholesterol. Since such inhibitors suppress blood cholesterol levels, the inhibitors are used as therapeutic agents for patients with hypercholesterolemia. As such HMG-CoA reductase inhibitors, natural products derived from microorganisms and semi-synthesized compounds derived from the natural products described above, and totally synthesized chemical compounds are all included. For instance, such compounds are (+)-(3R, 5R)-3,5-dihydroxy-7-[(1S,2S,6S,8S,8aR)-6-hydroxy-2-methyl-8-[(S)-2-methylbutyryloxy]-1,2,6,7,8,8a-hexahydro-1-naphtyl]heptanoic acid which is disclosed in Japanese Patent Publication (Kokai) Number Sho 57-2240 (U.S. Pat. No. 4,346,227) (hereinafter called pravastatin),
(+)-(1S,3R,7S,8S,8aR)-1,2,3,7,8,8a-hexahydro-3,7-dimethyl-8-[2-[(2R,4R)-tetrahydro-4-hydroxy-6-oxo-2H-pyran-2-yl]ethyl]-1-naphthyl (S)-2-methylbutyrate which is disclosed in Japanese Patent Publication (Kokai) Number Sho 57-163374 (U.S. Pat. No. 4,231,938) (hereinafter called lovastatin),
(+)-(1S,3R,7S,8S,8aR)-1,2,3,7,8,8a-hexahydro-3,7-dimethyl-8-[2-[(2R,4R)-tetrahydro-4-hydroxy-6-oxo-2H-pyran-2-yl]ethyl]-1-naphthyl 2,2-dimethylbutyrate which is disclosed in Japanese Patent Publication (Kokai) Number Sho 56-122375 (U.S. Pat. No. 4,444,784) (hereinafter called simvastatin),
(±)(3R*,5S*,6E)-7-[3-(4-fluorophenyl)-1-(1-methylethyl)-1H-indol-2-yl]-3,5-dihydroxy-6-heptenoic acid which is disclosed in Japanese Patent Publication (Kohyo) Number Sho 60-500015 (U.S. Pat. No. 4,739,073) (hereinafter called fluvastatin),
(3R,5S,6E)-7-[4-(4-fluorophenyl)-2,6-di(1-methylethyl)-5-methoxymethylpyridin-3-yl]-3,5-dihydroxy-6-heptenoic acid which is disclosed in Japanese Patent Publication (Kokai) Number Hei 1-216974 (U.S. Pat. No. 5,006,530) (hereinafter called rivastatin),
(3R,5S)-7-[2-(4-fluorophenyl)-5-(1-methylethyl)-3-phenyl-4-phenylaminocarbonyl-1H-pyrrol-1-yl]-3,5-dihydroxyheptanoic acid which is disclosed in Japanese Patent Publication (Kokai) Number Hei 3-58967 (U.S. Pat. No. 5,273,995) (hereinafter called atorvastatin), and (E)-3,5-dihydroxy-7-[4′-(4″-fluorophenyl)-2′-cyclopropyl-quinolin-3′yl]-6-heptenoic acid which is disclosed in Japanese Patent Publication (Kokai) Number Hei 1-279866 (U.S. Pat. No. 5,854,259 and U.S. Pat. No. 5,856,336) (hereinafter called pitavastatin), or
(+)-(3R,5S)-7-[4-(4-fluorophenyl)-6-isopropyl-2-(N-methyl-N-methanesulfonylamino)pyridin-5-yl]-3,5-dihydroxy-6(E)-heptenoic acid which is disclosed in Japanese Patent Publication (Kokai) Number Hei 5-178841 (U.S. Pat. No. 5,260,440) (hereinafter called rosuvastatin). In addition, an HMG-CoA reductase inhibitor, which is one component of the medicinal composition of the present invention, refers to other HMG-CoA reductase inhibitors described in the disclosed patents described above.

Planar chemical structures of representative HMG-CoA reductase inhibitors are shown below:

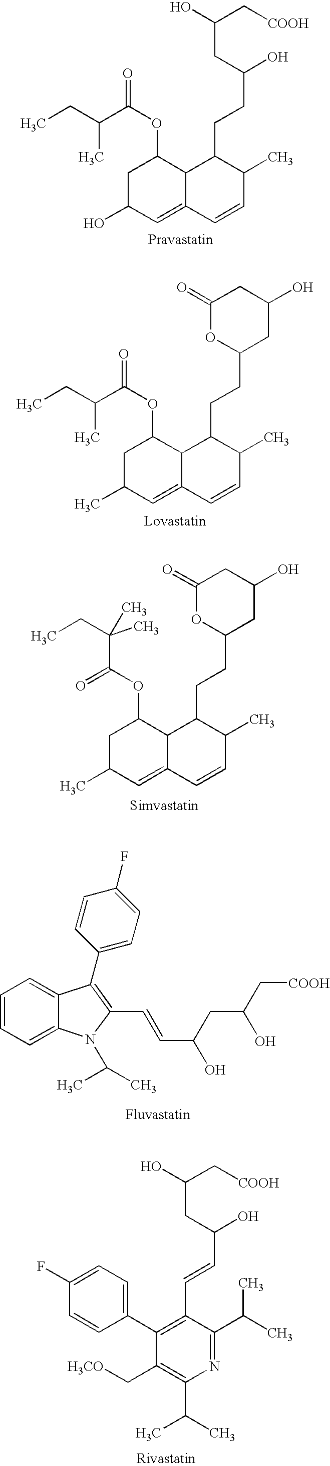

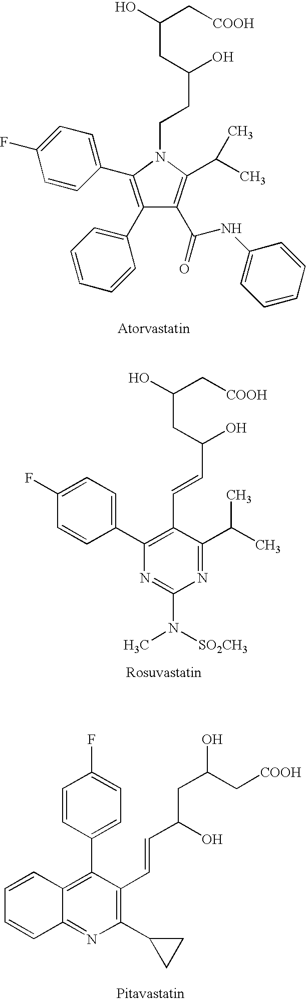

Furthermore, “bile acids” are, for example, ursodeoxycholic acid, chenodeoxycholic acid, deoxycholic acid, cholic acid, extracts from bile such as bile salts or bile extract, or the like; and bile of animals such as bear bile and the like, or gallstones of animals such as ox gallstone or the like, and preferably is ursodeoxycholic acid.

In the present invention, each active ingredients described above may be present as pharmacologically acceptable salts thereof, and
in the case that the active ingredients present a basic functional group, such salts are, for example, a hydrohalide such as hydrofluoride, hydrochloride, hydrobromide, hydroiodide, or the like; an inorganic acid salt such as a nitrate, a perchlorate, a sulfate, a phosphate, or the like; a lower organic sulfonate such as methanesulfonate, trifluoromethanesulfonate, ethanesulfonate, or the like; an arylsulfonate such as benzenesulfonate p-toluenesulfonate, or the like; an amino acid salt such as ornithine salt, glutamate, or the like; and carboxylic acid salt such as a fumarate, a succinate, a citrate, a tartrate, an oxalate, a maleate, or the like,
in the case that the active ingredients present an acidic functional group, such salts are, for example, an alkaline metal salt such as sodium salt, potassium salt, lithium salt, or the like; an alkaline earth metal salt such as calcium salt, magnesium salt, or the like; a metal salt such as an aluminium salt, an iron salt, a zinc salt, a copper salt, a nickel salt, cobalt salt, or the like; an amine salt, for example, an inorganic amine salt such as ammonium salt, an organic amine salt such as t-octylamine salt, dibenzylamine salt, morpholine salt, glucosamine salt, phenylglycine alkyl ester salt, ethylenediamine salt, N-methylglucamine salt, guanidine salt, diethylamine salt, triethylamine salt, dicyclohexylamine salt, N,N′-dibenzylethylenediamine salt, chloroprocaine salt, procaine salt, diethanolamine salt, N-benzylphenethylamine salt, piperazine salt, tetramethylammonium salt, tris(hydroxymethyl)aminomethane salt, or the like. For instance, in the case of pravastatin, a preferable salt is pravastatin sodium, and for instance, in the case of atorvastatin, a preferable salt is atorvastatin calcium salt hydrate.

In the case that each active ingredient involved forms a hydrate or solvate, such hydrates or solvates are included in the medicinal compositions of the present invention.

“Lower blood lipid levels” in the present invention means reducing blood lipid levels to clinically significant values, that is, reducing blood triglyceride levels, reducing blood LDL levels, or reducing blood total cholesterol levels. Thus the medicinal compositions of the present invention are useful as pharmaceutical agents to treat diseases caused by high blood lipid levels (for example, hypercholesterolemia and atherosclerosis, and the like).

HMG-CoA reductase inhibitors used as an active ingredient in the medicinal composition of the present invention, for example, pravastatin, lovastatin, simvastatin, fluvastatin, rivastatin, atorvastatin, pitavastatin, or rosuvastatin, can be easily prepared according to the methods described hereinafter in Japanese Patent Publication (Kokai) Number Sho 57-2240 (U.S. Pat. No. 4,346,227), Japanese Patent Publication (Kokai) Number Sho 57-16337 (U.S. Pat. No. 4,231,938), Japanese Patent Publication (Kokai) Number Sho 56-122375 (U.S. Pat. No. 4,444,784), Japanese Patent Publication (Kohyo) Number Sho 60-500015 (U.S. Pat. No. 4,739,073), Japanese Patent Publication (Kokai) Number Hei 1-216974 (U.S. Pat. No. 5,006,530), Japanese Patent Publication (Kokai) Number Hei 3-58967 (U.S. Pat. No. 5,273,995), Japanese Patent Publication (Kokai) Number Hei 1-279866 (U.S. Pat. No. 5,854,259and U.S. Pat. No. 5,856,336), or Japanese Patent Publication (Kokai) Number Hei 5-178841 (U.S. Pat. No. 5,260,440).

Of the bile acids, ursodeoxycholic acid can be easily obtained, since the specifications are disclosed in “The Japanese Pharmacopoeia (JP) 14th Edition”. Other bile acids can be easily obtained as commercially available products.

The medicinal compositions of the present invention contain an HMG-CoA reductase inhibitor and bile acids as essential active ingredients and additive agents may be contained for the formulation, as required. In addition, other active ingredients included in the medicinal compositions are not particularly restricted provided that they have no adverse effects when they are co-administered with the HMG-CoA reductase inhibitor and bile acids contained in the medicinal composition. The preferable medicinal composition is restricted to an HMG-CoA reductase inhibitor and bile acids alone as active ingredients and may contain additive agents and base agents for its formulation as required.

The concrete preparations of the medicinal composition of the present invention are, for example, tablets, granules (including powders), capsules, liquids and solutions (including syrups), and the like. These preparations are prepared by conventionally known methods disclosed in “The Japanese Pharmacopoeia (JP)” or the like using additive agents and bases that are suitable for each preparation, as necessary.

In each preparation described above, various conventionally used additive agents suitable for each preparation may also be used.

For example, in the case of tablets, diluents such as lactose, crystalline cellulose, or the like, stabilizers such as magnesium aluminometasilicate, magnesium oxide, or the like, coating agents such as hydroxypropylcellulose, or the like, and lubricants such as magnesium stearate, or the like may be used.

In the case of granules and capsules, diluents such as lactose, purified sucrose, or the like, stabilizers such as magnesium aluminometasilicate, magnesium oxide, or the like, adsorbents such as corn starch, or the like, and binders such as hydroxypropylcellulose, or the like may be used.

In each preparation described above, disintegrants such as crospovidone, or the like; surfactants such as polysorbate, or the like; adsorbents such as calcium silicate, or the like; colouring agents such as red ferric oxide, caramel, or the like; stabilizers such as sodium parahydroxybenzoate, or the like; pH modifiers; flavours, or the like, may be added if necessary.

In the present invention, “co-administration” means methods of administration of 2 or more active ingredients to humans simultaneously, or independent administration of 2 or more active ingredients described above at a certain time interval.

When the active ingredients of the present invention are administered, each active ingredient of the medicinal composition may be administered simultaneously or separately at a certain time

“Administration simultaneously” described above includes administration of each active ingredient at a pharmacologically acceptable time interval, in addition to administration of all active ingredients at the same time. There is no restriction provided that their pharmaceutical preparations are to be taken at roughly the same time. Nevertheless, it is favourable to take the 2 active ingredients as a single pharmaceutical preparation.

“Separate administration of 2 or more active ingredients described above at a certain time interval” described above has no restriction provided that their available pharmaceutical preparations are to be taken independently at different times. For instance, it indicates that first one active ingredient is administered, and then at after a defined time delay, the other active ingredient is administered.

Furthermore, in the case that the medicinal composition contains 3 or more active ingredients, “simultaneous administration or separate administration at certain time intervals” includes all cases wherein all active ingredients contained in the medicinal composition are taken simultaneously, each active ingredient is taken separately at certain time delays, 2 or more active ingredients contained in the medicinal composition are simultaneously taken and the rest of them are separately taken at certain time intervals, or 2 or more active ingredients in the medicinal composition are simultaneously taken and the rest of them are simultaneously taken after a certain time delay.

Subjects in whom the medicinal composition of the present invention can be administered are mammals, for instance, humans, dogs, cats, rabbits, oxen, horses, sheep, and pigs, and preferably humans and dogs, and more preferably humans.

Since the medicinal composition of the present invention comprising an HMG-CoA reductase inhibitor and bile acids exerts remarkable lowering effects on blood lipids, the medicinal compositions of the present invention are useful as pharmaceutical agents to prevent or to treat diseases caused by high blood lipid levels (for example, hypercholesterolemia, atherosclerosis, and the like).

In the present invention, the dosage of. HMG-CoA reductase inhibitors varies depending on the types of HMG-CoA reductase inhibitors used, the formulations, and the like. It is usual to administer 0.015-3.5 mg/kg per day and preferably 0.08-3.0 mg/kg per day to a mammal. For example, for an adult human, it is usual to administer 1 mg-200 mg per day and preferably 5 mg-160 mg per day.

In the present invention, the dosage of bile acids is usually 0.15-84 mg/kg per day and preferably 1.5-34 mg/kg per day to a mammal. For example, for an adult human, it is usual to administer 10 mg-5,000 mg per day and preferably 100 mg-2000 mg per day.

In the case that the dosage form of the medicinal composition of the present invention is a solid dosage form, the weight percentages of the HMG-CoA reductase inhibitor contained in the medicinal composition is usually 0.005-3% and preferably 0.03 to 2% for simvastatin, and in the case of atorvastatin, the weight percentage is usually 0.01-5%, and preferably 0.05-3%. In addition, in the case of ursodeoxycholic acid, the weight percentage is usually 0.3-90%, and preferably 3-50%.

In the case that the dosage form of lipid lowering agent contained in the medicinal composition of the present invention is a liquid or solution, the content of the HMG-CoA reductase inhibitor, for example, in the case of simvastatin, contained in the medicinal composition is usually 0.005-5 mg/mL, and preferably 0.03-3 mg/mL, while in the case of atorvastatin, the content is usually 0.01-10 mg/mL, and preferably 0.05-5 mg/mL. Furthermore, in the case of ursodeoxycholic acid, the content is usually 1-100 mg/mL, and preferably 10-500 mg/mL.

### EXAMPLES

The present invention will further be exemplified in more detail by the Examples, and the like. However the scope of the present invention is not limited by these Examples.

### Example 1

### Tablets

(1) Compositions

| | 6 Tablets | 6 Tablets | 6 Tablets |
|---|---|---|---|
| | (mg) | (mg) | (mg) |
| Atorvastatin calcium | 20 | — | 10 |
| Simvastatin | — | 10 | 5 |
| Ursodeoxycholic acid | 300 | 300 | 300 |
| Magnesium oxide | 400 | 400 | 400 |
| Magnesium | 140 | 140 | 140 |
| aluminometasilicate | | | |
| Crystalline cellulose | 120 | 120 | 120 |
| Corn starch | 140 | 140 | 140 |
| Hydroxypropylcellulose | 60 | 60 | 60 |
| Croscarmellose sodium | 15 | 15 | 15 |
| Magnesium stearate | 25 | 25 | 25 |
| Glycerin triacetate | 6 | 6 | 6 |
| Lactose | A suitable | A suitable | A suitable |
| | amount | amount | amount |
| Total | 1,200 | 1,200 | 1,200 |

(2) Manufacturing Methods

Each active ingredient described above is weighed and the tablets are manufactured according to methods described in General Rules for Preparation (tablets) of the Japanese Pharmacopoeia.

### Example 2

### Granules

(1) Compositions

| | 3 Packages | 3 Packages | 3 Packages |
|---|---|---|---|
| | (mg) | (mg) | (mg) |
| Atorvastatin calcium | 20 | — | 10 |
| Simvastatin | — | 10 | 5 |
| Ursodeoxycholic acid | 300 | 300 | 300 |
| Magnesium oxide | 400 | 400 | 400 |
| Magnesium | 140 | 140 | 140 |
| aluminometasilicate | | | |
| Purified sucrose | 1400 | 1400 | 1400 |
| Extracted products from | 15 | 15 | 15 |
| stevia | | | |
| Corn starch | 1200 | 1000 | 1100 |
| Polysorbate 80 | 80 | 80 | 80 |
| Magnesium stearate | 25 | 25 | 25 |
| Lactose | A suitable | A suitable | A suitable |
| | amount | amount | amount |
| Total | 4,300 | 4,300 | 4,300 |

(2) Manufacturing Methods

Each active ingredient described above is weighed and the granules are manufactured according to methods described in General Rules for Preparation (granules) of the Japanese Pharmacopoeia.

### Example 3

### Capsules

(1) Compositions

| | 6 Capsules | 6 Capsules | 6 Capsules |
|---|---|---|---|
| | (mg) | (mg) | (mg) |
| Atorvastatin calcium | 20 | — | 10 |
| Simvastatin | — | 10 | 5 |
| Ursodeoxycholic acid | 300 | 300 | 300 |
| Magnesium oxide | 400 | 400 | 400 |
| Corn starch | 600 | 400 | 500 |
| Polysorbate 80 | 50 | 50 | 50 |
| Magnesium stearate | 25 | 25 | 25 |
| Lactose | A suitable | A suitable | A suitable |
| | amount | amount | amount |
| Capsule | 480 | 480 | 480 |
| Total | 2,300 | 2,300 | 2,300 |

(2) Manufacturing Methods

Each active ingredient described above is weighed and granules are manufactured according to methods described in General Rules for Preparation (granules) of the Japanese Pharmacopoeia. The granules are filled in hard capsules.

### Example 4

### Syrups

(1) Compositions

| | 60 mL (mg) | 60 mL (mg) | 60 mL (mg) |
|---|---|---|---|
| Atorvastatin calcium | 20 | — | 10 |
| Simvastatin | — | 10 | 5 |
| Ursodeoxycholic acid | 300 | 300 | 300 |
| Sodium benzoate | 240 | 240 | 240 |
| Citric acid | 60 | 60 | 60 |
| Sucrose | 1,500 | 1,500 | 1,500 |
| Conc. Glycerin | 1,800 | 1,800 | 1,800 |
| Polyvinylalcohol | 120 | 120 | 120 |
| Ethanol (95%) | 500 | 9,000 | 4,500 |
| Hydrochloric acid | A suitable | A suitable | A suitable |
| | amount | amount | amount |
| Sodium hydroxide | A suitable | A suitable | A suitable |
| | amount | amount | amount |
| Purified water | A suitable | A suitable | A suitable |
| | amount | amount | amount |

(2) Manufacturing Methods

Each active ingredient described above is weighed and the syrups are manufactured according to methods described in General Rules for Preparation (syrups) of the Japanese Pharmacopoeia. The syrups are kept in brown glass bottles.

### Test Examples

### Test Example 1

### Evaluation Studies of Blood Lipid Levels

(1) Test Substance

Simvastatin and atorvastatin calcium synthesized at Chemtech Labo., Inc. were used. Ursodeoxycholic acid was purchased from Mitsubishi Pharma Corporation and used.

(2) Animals

Male Beagle dogs of 5 months of age were purchased from Covance Research Products Inc. as the test animals and were used after accommodation breeding for approximately 5 months.

(3) Dosage form, Preparation of the Formulation, and Storage

The required amount of the test substance calculated based on the body weight of the test animals was weighed and filled in gelatin capsules (TORPAC Inc., ½ oz). After filling up, the capsules were placed in a box divided up for every animal and stored under freezing until use.

(4) Route of Administration and Administration Period

The capsules filled up with the test substance were orally administered to the test animals once daily via an oral gavage between 9:00-12:30. The test animals were fasted for 2-3 hrs prior to each administration. The administration period was 11 days.

(5) Preparation of the Test Samples

Approximately 10 mL of blood was collected from the cephalic vein of the dog on −14 and −7 day (the 1st and the 2nd week prior to the administration) as well as 4, 8, and 12 day after administration. The animals were fasted for approximately 18 hrs before collection of the blood.

The collected blood was placed in a test tube and allowed to stand at room temperature for 30 min to 1 hour. Then the blood was centrifuged (approximately 1,600 g, for 10 min) and the resultant serum obtained was used for assays.

(6) Test Procedure

Total cholesterol content was assayed by enzymatic assay method, while LDL was determined by chemically modified method. Clinical Laboratory System (TBA-120FR, Toshiba Medical Systems Corporation) was used in all of these determinations.

(7) Results

Relative values of blood concentrations of various lipids in animals treated with each dose of ursodeoxycholic acid, simvastatin, or atorvastatin calcium alone as well as their medicinal compositions described above were calculated against each converted average value calculated from that determined 2 weeks and one week before the treatment into 100.

The results are summarized in Tables 1 and Table 2. The values indicate average results calculated from 5 dogs per group.

TABLE 1

| | Changes in blood NOₓ levels (%) | | |
|---|---|---|---|
| | 4 Days after | 8 Days after | 12 Days after |
| Test substance (mg/Kg) | treatment | treatment | treatment |
| Ursodeoxycholic acid (100) | 100.3 | 99.4 | 95.4 |
| Simvastatin (1) | 94.8 | 94.1 | 92.4 |
| Simvastatin (1) + | 91.6 | 82.4 | 81.5 |
| Ursodesoxycholic acid | | | |
| (100) | | | |
| Atorvastatin calcium (2) | 90.2 | 94.1 | 91.7 |
| Atorvastatin calcium (2) + | 91.0 | 82.8 | 82.1 |
| Ursodeoxycholic acid (100) | | | |

TABLE 2

| | Changes in blood NOₓ levels (%) | | |
|---|---|---|---|
| | 4 Days after | 8 Days after | 12 Days after |
| Test substance (mg/Kg) | treatment | treatment | treatment |
| Ursodeoxycholic acid (100) | 94.8 | 95.0 | 94.0 |
| Simvastatin (1) | 83.9 | 90.4 | 81.3 |
| Simvastatin (1) + | 73.0 | 62.9 | 66.5 |
| Ursodeoxycholic acid (100) | | | |
| Atorvastatin calcium (2) | 82.4 | 82.1 | 83.6 |
| Atorvastatin calcium (2) + | 69.9 | 68.1 | 59.1 |
| Ursodeoxycholic acid (100) | | | |

Remarkable lowering effects on blood lipid levels were elicited following administration of the medicinal composition containing ursodeoxycholic acid and either atorvastatin or simvastatin.

Since the medicinal compositions comprising an HMG-CoA reductase inhibitor and bile acids of the present invention exert potent lowering effects on blood lipid levels, the medicinal compositions of the present invention are useful as preventive or therapeutic agents for diseases caused by high blood lipid levels (for example, hyperlipidemia, atherosclerosis, and the like).

1. A pharmaceutical composition comprising effective amounts of an HMG-CoA reductase inhibitor and bile acids for lowering blood lipid levels.

2. The pharmaceutical composition according to claim 1, in which the HMG-CoA reductase inhibitor is one or more HMG-CoA reductase inhibitors selected from the group consisting of pravastatin, lovastatin, simvastatin, fluvastatin, rivastatin, atorvastatin, pitavastatin, and rosuvastatin.

3. The pharmaceutical composition according to claim 1, in which the HMG-CoA reductase inhibitor is simvastatin.

4. The pharmaceutical composition according to claim 1, in which the HMG-CoA reductase inhibitor is atorvastatin.

5. The pharmaceutical composition according to claims 1 or 2, in which said bile acids are one or more bile acids selected from the group consisting of ursodeoxycholic acid, chenodeoxycholic acid, deoxycholic acid, cholic acid, bile salts, bile extract, bear bile, and ox gallstone.

6. The pharmaceutical composition according to claim 5, in which the bile acid is ursodeoxycholic acid.

7. The pharmaceutical composition according to claim 3, in which the bile acid is ursodeoxycholic acid.

8. The pharmaceutical composition according to claim 4, in which the bile acid is ursodeoxycholic acid.

9. The pharmaceutical composition of claim 1 in solid dosage form containing 0.005-3% HMG-CoA reductase inhibitor and 0.3-90% ursodeoxycholic acid.

10. The pharmaceutical composition of claim 1 in liquid or solution dosage form containing 0.005-5 mg/mL simvastatin or 0.01-10 mg/mL atorvastatin; and containing 1-100 mg/mL ursodeoxycholic acid.

11. A method for lowering blood lipid levels comprising administering effective amounts of an HMG-CoA reductase inhibitor and bile acids simultaneously or separately, to a mammal in need thereof.

12. The method according to claim 11 for preventing or treating diseases caused by high blood lipid levels.

13. The method according to claim 11 for preventing or treating hypercholesterolemia or atherosclerosis.

14. The method according to claim 11, comprising administering in one or more dosages, to an adult human in need thereof, a total of 1-200 mg HMG-CoA reductase inhibitor and 10-5,000 mg of bile acids.

15. The method according to claim 11, wherein the one or more HMG-CoA reductase inhibitor is selected from the group consisting of pravastatin, lovastatin, simvastatin, fluvastatin, rivastatin, atorvastatin, pitavastatin, and rosuvastatin; and the one or more bile acids is selected from the group consisting of ursodeoxycholic acid, chenodeoxycholic acid, deoxycholic acid, cholic acid, bile salts, bile extract, bear bile, and ox gallstone.

16. The method for lowering blood lipid levels comprising administering an effective amount of the pharmaceutical composition of claim 5.
